(19) United States
(12) Patent Application Publication
(10) Pub. No.: US 2015/0068538 A1
(43) Pub. Date: Mar. 12, 2015

(54) Oral Airway and Intubation Assisting Device

(71) Applicants: Trevor Wesolowski, Chicago, IL (US); Matthew Hanley, Sterling, VA (US); Zachary Stimart, McLean, VA (US); Nancy Rios, Chicago, IL (US); Catherine Santis, Chicago, IL (US); Juan C. Rodriguez Aranda, Chicago, IL (US); Johnny Villasenor-Arreola, Chicago, IL (US); Melissa T. Wardlow, Chicago, IL (US)

(72) Inventors: Trevor Wesolowski, Chicago, IL (US); Matthew Hanley, Sterling, VA (US); Zachary Stimart, McLean, VA (US); Nancy Rios, Chicago, IL (US); Catherine Santis, Chicago, IL (US); Juan C. Rodriguez Aranda, Chicago, IL (US); Johnny Villasenor-Arreola, Chicago, IL (US); Melissa T. Wardlow, Chicago, IL (US)

(21) Appl. No.: 14/481,523

(22) Filed: Sep. 9, 2014

Related U.S. Application Data
(60) Provisional application No. 61/875,717, filed on Sep. 10, 2013.
(60) Provisional application No. 61/982,348, filed on Apr. 22, 2014.

(51) Int. Cl. A61M 16/04

(52) U.S. Cl. CPC A61M 16/0493; A61M 16/0497
USPC 128/861

(57) ABSTRACT

An oral airway and intubation assisting device that protects a patient's teeth and includes a wedge adapted to redirect force from the patient's incisor teeth to the patient's maxilla, a block adapted to hold the patient's mouth open, and an airway guide adapted to prevent the tongue from closing the throat and maintain a clear passage for air. Additionally, the device may include a tube retainer adapted to secure the intubation tube against movement.

This application claims priority to provisional application Ser. No. 61/875,717 filed Sep. 10, 2013 to the extent allowed by law, and to provisional application Ser. No. 61/982,348 filed Apr. 22, 2014 to the extent allowed by law.

### BACKGROUND OF THE INVENTION

1. Field of the Invention

The present invention relates generally to the technical field of medical devices, and more particularly, to the technical field of oral airway and intubation assisting devices.

2. Description of the Prior Art

During surgeries and emergency medical situations, a doctor, nurse, or medical technician will often place a tube into a patient's trachea. The tube stabilizes the patient's airway, providing an unobstructed passage for air or other gases to the patient's lungs.

The process of placing the tube into a patient's trachea is called intubation. As typically carried out, the patient lies face up and the medical practitioner stands at the patient's head and uses their right hand to open the patient's mouth. The medical practitioner holds a device called a laryngoscope in their left hand. The laryngoscope includes a cylindrical handle attached perpendicularly to a curved metal blade, the arc of which approximates the curve of the patient's throat. By seating this blade against the patient's tongue and lower throat and lifting the tongue and lower throat forward and upward, the medical practitioner can create a straight line of sight into the patient's larynx. With this view, the medical practitioner uses their right hand to thread the intubation tube between the patient's vocal chords and into the trachea. The medical practitioner seats the tube in the person's trachea and withdraws the laryngoscope. Often, the practitioner will secure the tube against further movement with tape or a fixture device.

Before the intubation tube establishes a clear airway to the patient's lungs, the patient breathes with a mask that seals to their face. However, without an intubation tube in place, the patient's airway may be obstructed by the patient's tongue and tissues. To remedy this situation, a short tube called an oropharyngeal airway is placed in the patient's mouth. The oropharyngeal airway provides a passageway for air to the larynx, but not beyond. The oropharyngeal airway takes up a great deal of room in the patient's mouth and must be removed before beginning the intubation process. If the medical practitioner cannot seat the intubation tube in the patient's trachea, they will remove the laryngoscope and replace the oropharyngeal airway to establish breathing and then attempt intubation again.

Sometimes a medical practitioner will use the patient's upper teeth as a fulcrum for the laryngoscope blade, levering the patient's tongue and throat upward. While this method is incorrect, it is used occasionally and places large forces on the upper teeth, sometimes chipping or breaking the patient's upper teeth. The patient can inhale pieces of teeth, causing further complications. Additionally, the patient's upper lip may slip between their teeth and the laryngoscope blade causing the patient's lip to be cut or bruised.

U.S. Pat. No. 8,104,467 discloses a rapid orotracheal intubation guide that facilitates orotracheal intubation or direct orotracheal visualization without resting the device on the patient's tongue. During bronchoscopic intubation, the device automatically ejects the endotracheal tube from the device as the endotracheal tube is advanced coaxially over a bronchoscope previously placed through the guide. The device in patent '467 does not work with a traditional laryngoscope and must be removed in order to use the laryngoscope. The device in patent '467 also does not include external tube fixation and does not include force redirection from the patient's incisors to the mandible, because the device simply covers the patient's teeth.

U.S. Patent Application Publication 2007/0197876 discloses a dental guard for airway intubation that includes an upper mouth guard for the maxillary teeth attached to blocks or wedges for keeping the jaw open. Once intubation has been accomplished and the laryngoscope has been removed, the dental guard may be removed, may remain in the patient's jaw as a bite block, or may be replaced with another type of bite block. However, the dental guard in publication '876 does not allow access to the right side of the patient's mouth and impedes the use of a traditional laryngoscope.

U.S. Pat. No. 7,866,313 discloses an oral airway that facilitates tracheal intubation that includes a first component having a first guiding surface and a second component having a second guiding surface. The first component and the second component are removably coupled together such that the first guiding surface and the second guiding surface together define an interior passage through the oral airway that is dimensioned to direct a fiber-optic scope or an endotracheal tube extending through the interior passage for tracheal intubation. The first component and second component can then be decoupled for independent removal from the patient's mouth, without disrupting the endotracheal tube. The oral airway of patent '313 holds the patient's tongue back, however, it comprises two separate pieces and does not allow for the use of a laryngoscope.

The prior art to date does not disclose an oral airway and intubation assisting device that can be used with a laryngoscope, protects the patient's upper teeth, distributes force from the patient's incisor teeth to the mandible bone, holds the patient's mouth open, and maintains the patient's tongue up and to the side to maintain a clean airway. None of the prior art can be combined in a way to suggest these necessary modifications. There is no teaching, suggestion, or motivation that would have enabled a person of ordinary skill in the art to modify any prior art oral airway and intubation assisting device to arrive at the present invention.

It is a primary object of the present invention to provide an oral airway and intubation assisting device that holds the patient's mouth open to free up the medical practitioner's hand and to aid in visualization of the larynx.

Another object of the present invention is to provide an oral airway and intubation assisting device that maintains a clear airway by moving the patient's tongue up and to one side of the mouth.

Still another object of the present invention is to provide an oral airway and intubation assisting device that protects a patient's teeth from impact, force, or other damage by a laryngoscope or other oral devices.

Still another object of the present invention is to provide an oral airway and intubation assisting device that includes a wedge across the patient's teeth that prevents tooth damage by redirecting the force applied by the laryngoscope from the incisor teeth to the mandible bone.

Still another object of the present invention is to provide an oral airway and intubation assisting device that includes a wedge that holds the patient's lips away from the laryngoscope and its pinch points.

Still another object of the present invention is to provide an oral airway and intubation assisting device that keeps the patient's mouth unobstructed, thereby allowing concurrent use with a traditional laryngoscope without having to remove the device.

Still another object of the present invention is to provide an oral airway and intubation assisting device that can be left in the patient's mouth to prevent the patient from biting down on the tube.

Still another object of the present invention is to provide an oral airway and intubation assisting device that includes a clip to hold the endotracheal tube without having to tape the tube to the patient's face.

### SUMMARY OF THE INVENTION

The oral airway and intubation assisting device of the present invention comprises a mouth guard to assist in the intubation that provides an airway passage and mouth opening wedge. The mouth guard comprises at least one member adapted to protect the patient's teeth, at least one block adapted to maintain the patient's mouth open, and at least one airway guide adapted to allow air to flow past the patient's tongue. The wedge protects the patient's teeth from the high force loads imposed by a laryngoscope and redirects those forces to the patient's maxilla, the upper jaw bone structure. The wedge also keeps the patient's lip clear of the upper teeth. Additionally, the mouth guard may include a tube retainer that secures the intubation tube in the patient's mouth.

### BRIEF DESCRIPTION OF THE DRAWINGS

The present invention is further described with reference to the accompanying drawings in which:

FIG. 1 is a top perspective view of a first embodiment of the oral airway and intubation assisting device of the present invention that includes a first embodiment of a tube retainer.

FIG. 2 is a front perspective view of the first embodiment of the oral airway and intubation assisting device of the present invention that includes the first embodiment of the tube retainer.

FIG. 3 is a front perspective view of a second embodiment of the oral airway and intubation assisting device of the present invention.

FIG. 4 is a front plan view of a third embodiment of the oral airway and intubation assisting device of the present invention.

FIG. 5 is a top plan view of the third embodiment of the oral airway and intubation assisting device of the present invention.

FIG. 6 is a top perspective view of a fourth embodiment of the oral airway and intubation assisting device of the present invention that includes a second embodiment of a tube retainer.

FIG. 7 is a top perspective view of a fifth embodiment of the oral airway and intubation assisting device of the present invention that includes a third embodiment of a tube retainer.

FIG. 8 is a top perspective view of the fifth embodiment of the oral airway and intubation assisting device of the present invention that includes a fourth embodiment of a tube retainer.

FIG. 9 is a top perspective view of the fourth embodiment of the oral airway and intubation assisting device of the present invention that includes a fifth embodiment of a tube retainer.

FIG. 10 is a top perspective view of the fourth embodiment of the oral airway and intubation assisting device of the present invention that includes the fifth embodiment of the tube retainer with an intubation tube in place.

FIG. 11 is a top perspective view of the fifth embodiment of the oral airway and intubation assisting device of the present invention that includes a sixth embodiment of a tube retainer.

FIG. 12 is a top perspective view of the fifth embodiment of the oral airway and intubation assisting device of the present invention that includes the sixth embodiment of the tube retainer twisted into a serpentine structure.

### DETAILED DESCRIPTION OF THE ILLUSTRATED EMBODIMENTS

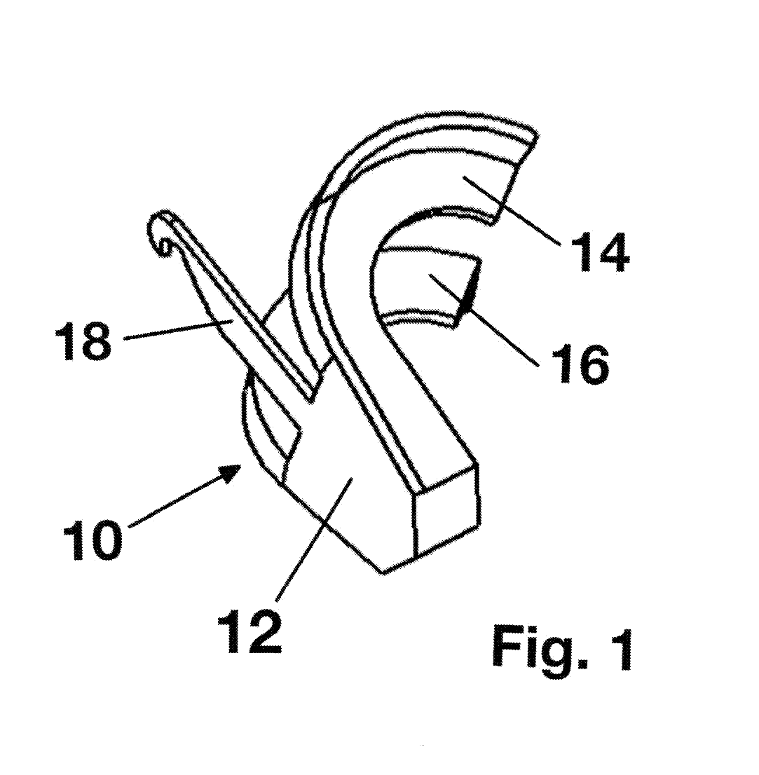

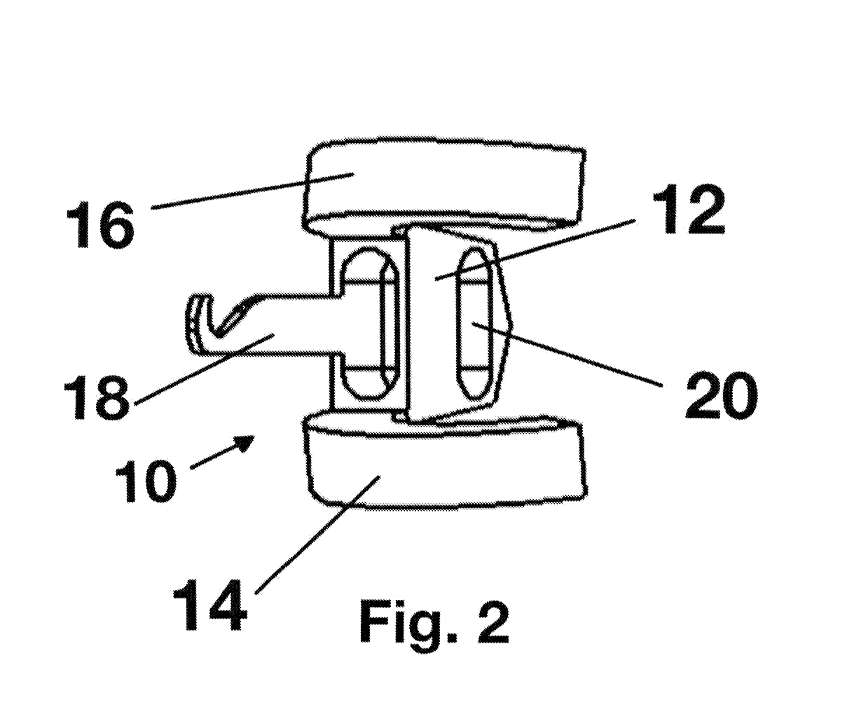

As illustrated in FIGS. 1 and 2 the oral airway and intubation assisting device of a first embodiment of the present invention comprises a mouth guard 10 having a block section 12 connecting a first curved member 14 and a second curved member 16. Each of the curved members 14, 16 is attached to the block section 12. A first embodiment of a tube retainer 18 is attached to the block section 12. An airway maintenance guide 20 is integrated into the block section 12.

In this broad embodiment, the present invention includes at least one member 14, 16 shaped to protect the teeth, connected to at least one block 12. The members 14, 16 that protect the teeth are shaped such that the forces applied to the teeth are redirected to the maxilla. Attached to the block 12 or the members 14, 16 shaped to protect the teeth is a portion 20 that allows air past the tongue. Additionally, the embodiment may include a tube retainer 18 to retain the intubation tube.

In further detail, still referring to the first embodiment shown in FIGS. 1 and 2, the airway maintenance guide 20 is sufficiently wide for the free passage of air, such as about three-eighths of an inch wide. The first curved member 14 and second curved member 16 are sufficiently long enough to cover at least the incisors, such as about one inch long. The tube retainer 18 is sufficiently thin to allow it to flex out of the way of the laryngoscope, such as about one-eighth inch wide, and is sufficiently long enough, such as about two inches long, to project out of the patient's mouth. The mouth guard 10 can be manufactured in a variety of sizes to accommodate different sized patients.

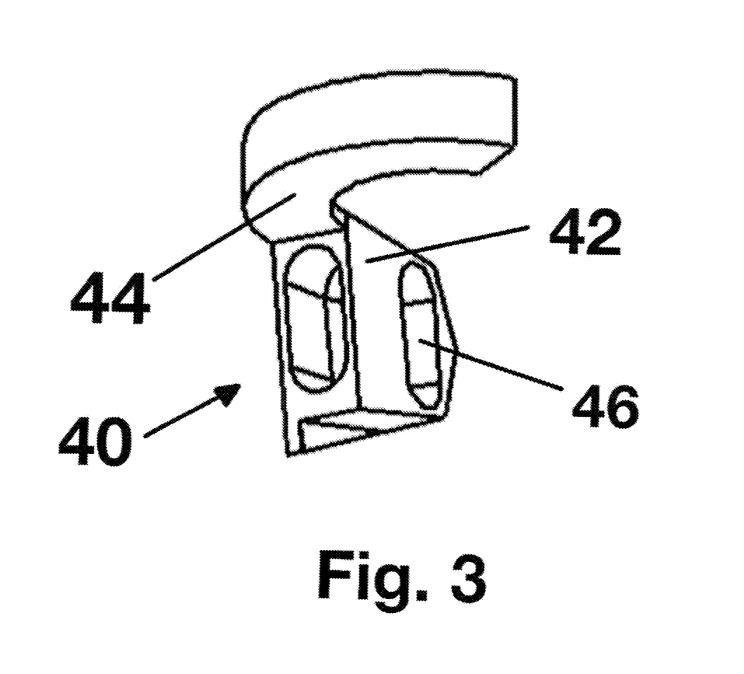

Referring to FIG. 3, a second embodiment of the present invention comprises a mouth guard 40 having a single block section 42 connected to a single curved member 44 to provide protection to only one set of teeth and leave more free space in the patient's mouth. An airway maintenance guide 46 is integrated into the block section 42. The broad, second embodiment of the present invention includes at least one member 44 shaped to protect the teeth, connected to at least one block section 42. The block section 42 may include an airway maintenance guide 46 through the mouth, or other features that allow air past the tongue. The second embodiment of the present invention may include a feature to retain the intubation tube.

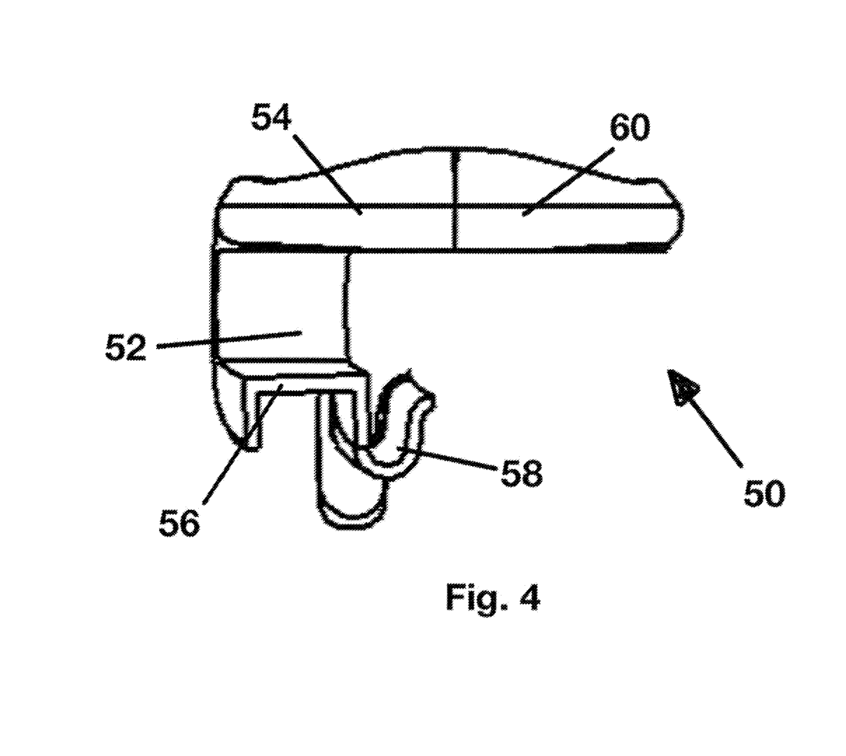

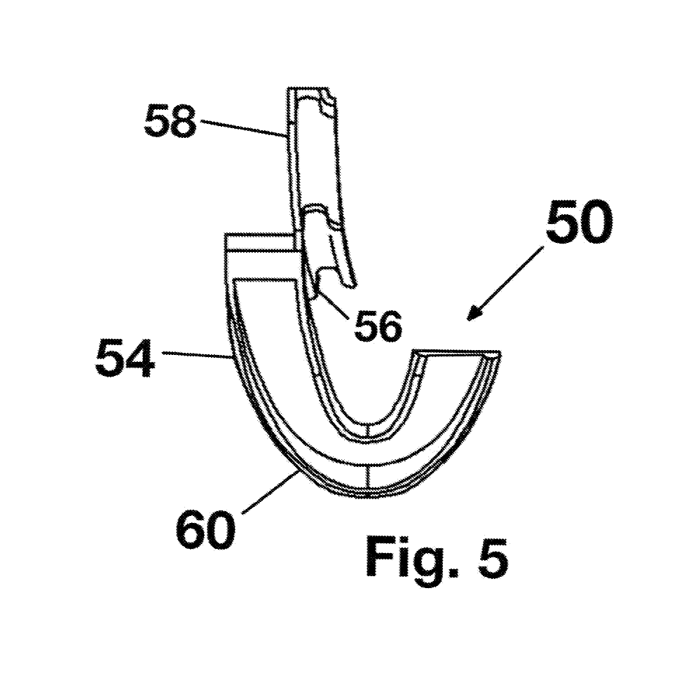

A broad, third embodiment of the present invention, shown in FIGS. 4 and 5, comprises a mouth guard 50 having a block section 52 connecting a single curved member 54 and a support member 56 that maintain the patient's mouth open. The single curved member 54 is sufficiently long enough to cover at least the patient's incisor teeth with a tray profile shaped to secure the single curved member 54 to the patient's upper teeth. The single curved member 54 includes a wedge 60 along its front face. The wedge 60 projects from the front face of the single curved member 54 far enough to displace the patient's upper lips upward and redirect the force of the laryngoscope to the patient's maxilla. The support member 56 is long enough to locate the mouth guard 50 to the patient's lower teeth with a tray profile to secure the support member 56 to the patient's lower teeth. The mouth guard 50 can be manufactured in a variety of sizes to accommodate differently sized patients.

An airway maintenance guide 58 is connected to at least one of the block section 52 and the support member 56 and includes a clear cross-sectional area that is sufficiently wide for the free passage of air. The airway maintenance guide 58 curves to follow the contour of a patient's throat, with a profile shaped to push the patient's tongue to the left. The airway maintenance guide 58 is located sufficiently far from the single curved member 54 so that the patient's mouth is clear for the insertion and placement of the laryngoscope. The third embodiment of the present invention may include a feature to retain the intubation tube.

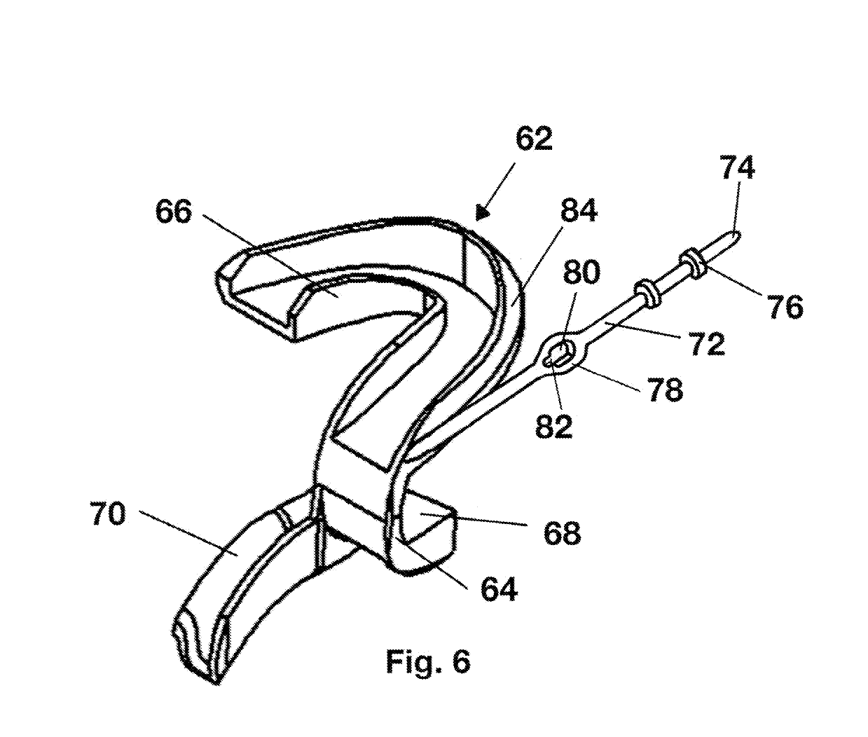

As illustrated in FIG. 6, a fourth embodiment of the present invention comprises mouth guard 62 having a block section 64 connecting a single curved member 66 and a support member 68. The single curved member 66 and the support member 68 are attached to the block section 64. An airway maintenance guide 70 is connected to the block section 64 and the support member 68. A second embodiment of a tube retainer 72 is attached to the single curved member 66.

In further detail, still referring to the embodiment of FIG. 6, the airway maintenance guide 70 includes a clear cross-sectional area that is sufficiently wide for the free passage of air. The airway maintenance guide 70 curves to follow the contour of a patient's throat, with a profile shaped to push the patient's tongue to the left. The airway maintenance guide 70 is located sufficiently far from the single curved member 66 so that the patient's mouth is clear for the laryngoscope. The tube retainer 72 comprises a long thin member 74, with at least one enlarged area 76 and an aperture 78. The long thin member 74 is small enough in cross-section that it is flexible and the enlarged areas 76 are of sufficient size to pass through the wide portion 80 of the aperture 78 yet catch in the thin portion 82 of the aperture 78 when member 74 is looped around and the top end of member 74 is inserted into aperture 78.

Referring again to FIG. 6, the single member 66 is sufficiently long enough to cover at least the patient's incisor teeth with a tray profile shaped to secure the single curved member 66 to the patient's upper teeth. The single curved member 66 includes a wedge 84 along its front face. The wedge 84 projects from the front face of the single curved member 66 far enough to displace the patient's upper lips upward and redirect the force of the laryngoscope to the patient's maxilla. The support member 68 is long enough to locate the mouth guard 62 to the patient's lower teeth with a tray profile to secure the support member 62 to the patient's lower teeth. The mouth guard 10 can be manufactured in a variety of sizes to accommodate differently sized patients.

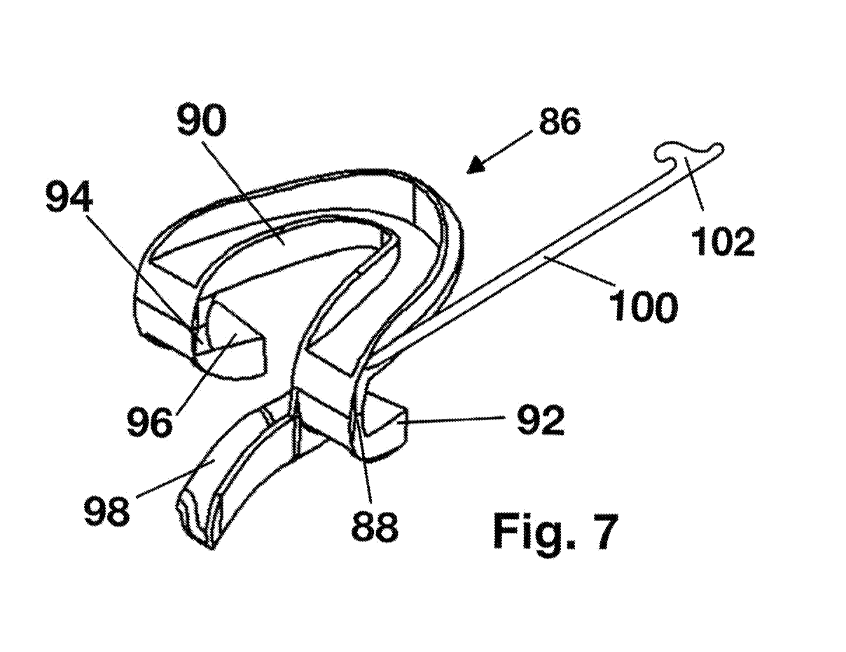

Referring to FIG. 7, a fifth embodiment of the present invention comprises mouth guard 86 having a first block section 88 connecting a larger first curved member 90 and a smaller second curved member 92. Additionally, a second block section 94 connects the larger first curved member 90 to a smaller third curved member 96. An airway maintenance guide 98 is attached to the first block section 88 and the second curved member 92. A third embodiment of a tube retainer 100 is connected to the first curved member 90.

In further detail, still referring to the fifth embodiment shown in FIG. 7, the second block section 94 and third curved member 96 provide additional engagement with the patient's lower teeth. The tube retainer 100 includes at least one hook 102 and is sufficiently thin so as to be flexible. The hook 102 has a diameter matching the thickness of the tube retainer 100 so that the hook 102 catches on and holds the tube retainer 100.

The various embodiments of the present invention can include various embodiments of the tube retainer. A first embodiment of the tube retainer 18 was shown in FIGS. 1 and 2. A second embodiment of the tube retainer 72 was shown in FIG. 6, and a third embodiment of the tube retainer 100 was shown in FIG. 7. Any embodiment of the tube retainer may be included in any embodiment of the present invention.

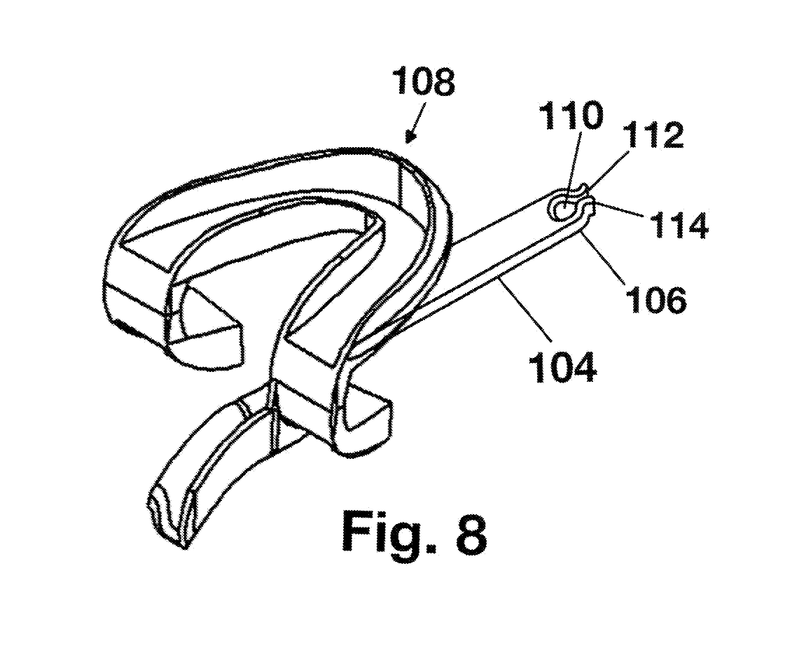

A fourth embodiment of the tube retainer is shown in FIG. 8 attached to the fifth embodiment of the mouth guard 86 of the present invention. The tube retainer 104 includes a flexible clip 106 that extends in front of the mouth guard 86. Clip 106 has a hollow opening 110, through which the intubation tube passes, and two prongs 112, 114. The intubation tube is pushed through and past the two prongs 112, 114 and is secured in opening 110 through tension applied by the two prongs 112, 114.

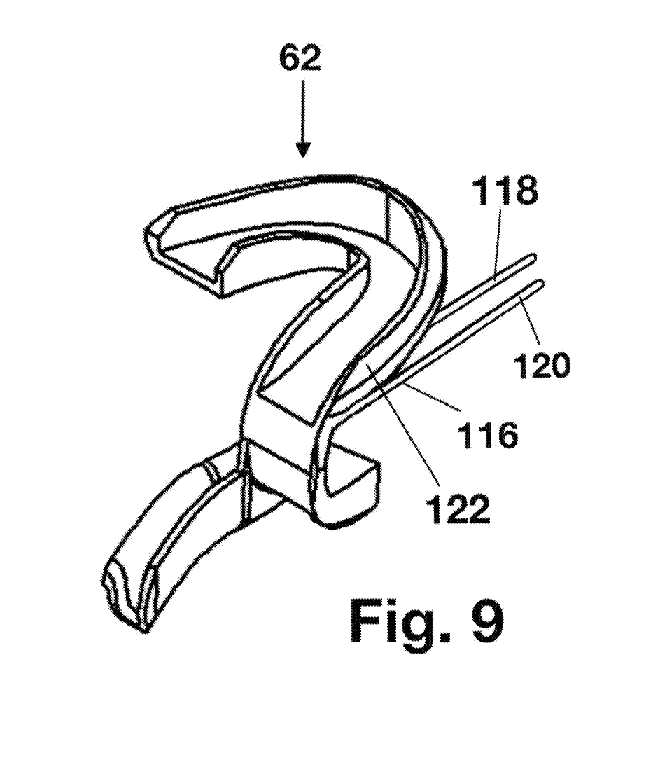

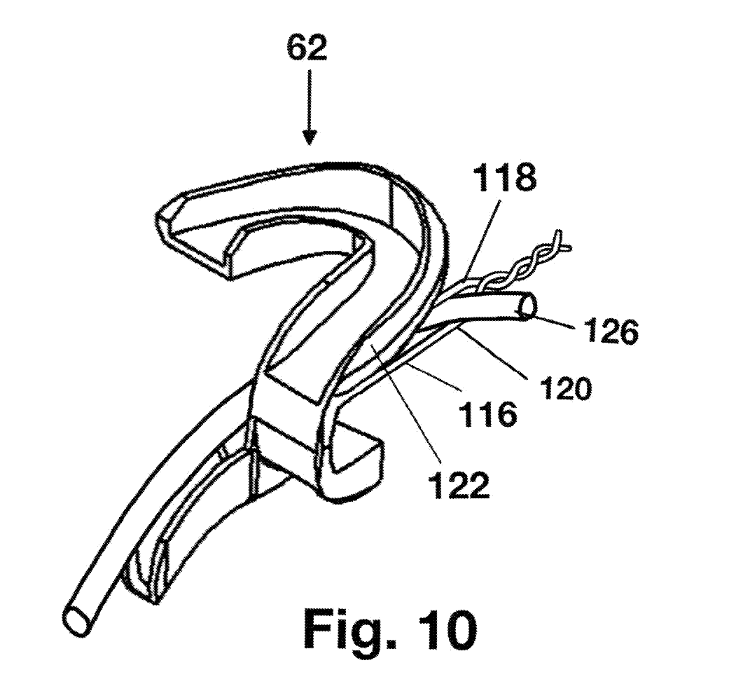

A fifth embodiment of the tube retainer is shown in FIGS. 9 and 10 attached to the fourth embodiment of the mouth guard 62 of the present invention. The tube retainer 116 includes two flexible strings 118, 120, shown in FIG. 9, that extend from the underside of the superior portion 122 of the mouth guard 62. Once the intubation tube 126 is inserted in the patient's throat, strings 118, 120 are wrapped around the intubation tube 126 and twisted around each other, shown in FIG. 10, securing the intubation tube 126 to the mouth guard 62.

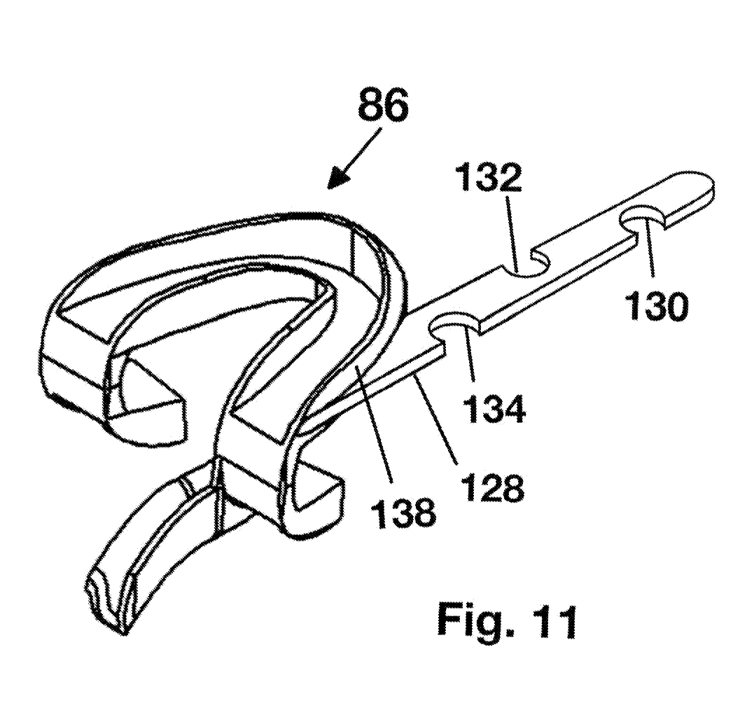

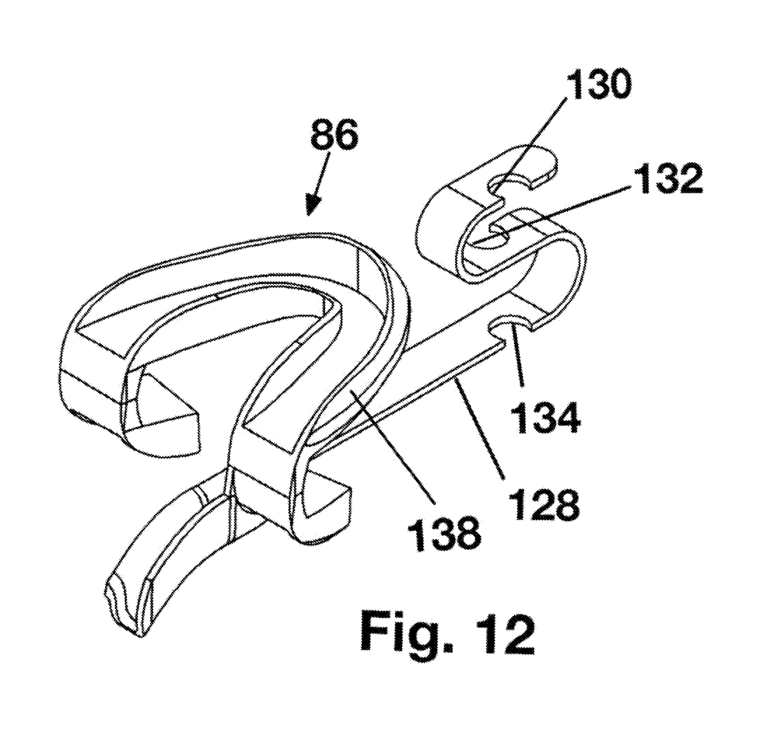

A sixth embodiment of the tube retainer is shown in FIGS. 11 and 12 attached to the fifth embodiment of the mouth guard 86 of the present invention. The tube retainer 128 includes three semi-circular apertures 130, 132, 134 that extend forward of the mouth guard 86 at the underside of the superior portion 138 of the mouth guard 86. The tube retainer 128 is twisted into a serpentine structure, as shown in FIG. 12, and the intubation tube is first placed into aperture 134, woven back through aperture 132, and passed through aperture 130. The twisted serpentine structure of tube retainer 128 provides tension among apertures 130, 132, 134 to secure the intubation tube to mouth guard 86.

The oral airway and intubation assisting device of the present invention may be made of plastic or other sufficiently rigid material, such as silicone, urethane, and the like. Further, the various parts of the oral airway and intubation assisting device may be made from different materials. Further still, the various parts may be made from more than one material.

The foregoing description of illustrated embodiments of the invention has been presented for purposes of illustration and description, and is not intended to be exhaustive or to limit the invention to the precise forms disclosed. The description was selected to best explain the principles of the invention and practical application of these principles to enable others skilled in the art to best utilize the invention in various embodiments and various modifications as are suited to the particular use contemplated. It is intended that the scope of the invention not be limited by the specification, but be defined by the claims set forth below.

What is claimed is:

1. An oral airway device adapted to assist in intubation of a patient, comprising:
a. at least one block, said block adapted to maintain the patient's mouth open;
b. a first curved member attached to said block, said first curved member adapted to be applied to and protect the teeth of the patient; and
c. a guide integral to one of said block and said first curved member, said guide adapted to allow air to pass the tongue of the patient.

2. The oral airway device of claim 1, wherein the guide is integrally formed within said block.

3. The oral airway device of claim 1, wherein the first curved member is adapted to protect the upper teeth of the patient.

4. The oral airway device of claim 1, further comprising a second curved member attached to said block, the second curved member adapted to protect the bottom teeth of the patient.

5. The oral airway device of claim 1, further comprising a tube retainer attached to one of said block and said first curved member, the tube retainer adapted to secure an intubation tube to the oral airway device.

6. The oral airway device of claim 1, wherein the first curved member includes a wedge that projects from a front face of the first curved member, the wedge adapted to displace the patient's upper lips upward and redirect force applied to the patient's teeth to the patient's maxilla.

7. The oral airway device of claim 1, wherein the guide is at least three-eighths of an inch wide.

8. The oral airway device of claim 1, wherein the first curved member is adapted to be at least long enough to cover the patient's incisor teeth.

9. The oral airway device of claim 8, wherein the first curved member is at least one inch long.

10. The oral airway device of claim 4, wherein the second curved member is adapted to be at least long enough to cover the patient's incisor teeth.

11. The oral airway device of claim 10, wherein the second curved member is at least one inch long.

12. The oral airway device of claim 5, wherein the tube retainer is adapted to be at least long enough to project out of the patient's mouth and is adapted to flex out of the way of a laryngoscope.

13. The oral airway device of claim 12, wherein the tube retainer is at least two inches long.

14. The oral airway device of claim 5, wherein the tube retainer includes a hook on a first end, the hook adapted to secure the intubation tube to the oral airway device.

15. The oral airway device of claim 1, wherein the guide includes a clear cross-sectional area adapted to allow the passage of air.

16. The oral airway device of claim 1, wherein the guide curves to follow the contour of the patient's throat and is adapted to push the patient's tongue to the left.

17. The oral airway device of claim 1, wherein the guide is spaced a distance from the first curved member to provide a path for a laryngoscope to be placed in the patient's mouth.

18. The oral airway device of claim 5, wherein:
a. the tube retainer comprises a flexible, long, thin member that includes at least one enlarged area and an aperture;
b. said aperture having a wide portion and a thin portion; and
c. said at least one enlarged area adapted to pass through the wide portion of the aperture and catch in the thin portion of the aperture.

19. The oral airway device of claim 1, further comprising a second block attached to the first curved member, the second block connecting the first curved member to a third curved member, said second block and third curved member adapted to provide additional engagement with the patient's lower teeth.

20. The oral airway device of claim 14, wherein the internal diameter of the hook is the same as the external diameter of the tube retainer, the hook adapted to catch onto the tube retainer.

21. The oral airway device of claim 5, wherein the tube retainer includes a clip that extends in front of the oral airway device, said clip having a hollow opening adapted to receive an the intubation tube and two prongs adapted to secure the intubation tube in the opening through tension provided by the two prongs.

22. The oral airway device of claim 5, wherein the tube retainer includes two flexible members that extend from an underside of a superior portion of the oral airway device, said two flexible members adapted to wrap around the intubation tube and twist around each other to secure the intubation tube to the oral airway device.

23. The oral airway device of claim 5, wherein the tube retainer includes at least a first aperture, a second aperture, and a third aperture that extend from an underside of a superior portion of the oral airway device, said tube retainer adapted to be formed into a serpentine structure such that the intubation tube is passed through the third aperture, woven back through the second aperture, and passed through the first aperture, said serpentine structure adapted to provide tension among the first aperture, second aperture, and third aperture to secure the intubation tube to the oral airway device.

24. The oral airway device of claim 1, wherein the oral airway device is made of a rigid material.

25. The oral airway device of claim 24, wherein the rigid material is at least one of plastic, silicone, and urethane.
